(19) United States
(12) Patent Application Publication
(10) Pub. No.: US 2023/0397871 A1
(43) Pub. Date: Dec. 14, 2023

(54) SENSING CONNECTOR ASSEMBLY AND SYSTEM COMPRISING SAID SENSING CONNECTOR ASSEMBLY

(71) Applicant: Viturin Ltd, London (GB)

(72) Inventors: Mathilde CRETTAZ, Lausanne (CH); Pascal HECK, Rolle (CH); Urs RUGGABER, St. Urban (CH)

(21) Appl. No.: 18/032,323

(22) PCT Filed: Oct. 15, 2020
(86) PCT No.: PCT/IB2020/059700

§ 371 Date: Apr. 17, 2023

(51) Int. Cl. A61B 5/274; A61B 5/256; A61B 5/282; A61B 5/332; A61B 5/00

(52) U.S. Cl. CPC A61B 5/274; A61B 5/256; A61B 2562/227; A61B 5/332; A61B 5/6804; A61B 5/282

(57) ABSTRACT

Sensing connector assembly (100) for providing an electrical connection between an electrical contact (12) of a sensor device (10) and an electrically conductive path (220) on a wearable fabric (200), comprising: a first connecting part (20) comprising an opening (32) configured to receive the sensor device (10); a second connecting part (40) configured to mechanically cooperate with the first connecting part (20) such that the first and second connecting parts (20, 40) can be fastened on each side of the wearable fabric (200) and extend in a connection plane (P). The first connecting part (20) comprises a first connecting rim (34) at the periphery of the opening (32) and configured to cooperate with sensor device (10) such as to attach the sensor device (10) to the first connecting part (20) and such that the electrical contact (12) exerts a predetermined force in a direction substantially orthogonal to the connection plane (P) and against the first side (201) of the wearable fabric (200), when the second connecting part (40) is fastened to the first connecting part (20).

### TECHNICAL DOMAIN

The present disclosure concerns a sensing connector assembly for providing an electrical connection between an electrical contact of a sensor device and an electrically conductive path on a first side of a wearable fabric. The present disclosure further concerns a system comprising a wearable fabric including at least an electrically conductive path and at least one sensing connector assembly.

### RELATED ART

Electric equipment to be worn by a user (person, namely a human being or an animal) can be used to monitor or measure physical parameters in relation with the user's body or in relation with the environment of the user. As non-limitative examples, such electric equipment can be an electrode of an electrocardiogram recorder or an electrode of a EIT (Electrical impedance tomography) monitoring device, a SpO₂ sensor (oxygen saturation sensor), a blood pressure sensor, a temperature sensor (body temperature sensor or air temperature sensor), gas concentration sensor (CO₂, CO, O₂ . . . ) or any sensor that can detect or measure vital sign parameters of the wearer body or physical and/or chemical parameter that can be detected outside the body. Such electric equipment can comprise a central unit exchanging information with other electric equipment placed elsewhere on the user.

The electronic equipment is subject to several constraints, such as: user motion, the ability to remain at a given location on the user's body, maintain the electrical connection between parts of the measurement units, being adapted to operate during a long time period and being resistant to humidity, including body sweat.

In some cases, an ambulatory electrocardiography device can be used for monitoring electrical activity of the user for at least 24 to 72 hours. Such device can record electrical signals from the heart via a series of electrodes contacting the user's skin. The electrodes are typically attached to the user's chest using adhesive electrodes and/or suction clip electrodes. The electrodes are connected via cables to a central electronic unit. The central electronic unit can be attached to a user's belt or hung around the neck. Such ambulatory electrocardiography device presents some drawbacks, namely: requiring a full body shave and causing possible skin dermatitis, signal-quality issues, frequent electrode detachments causing ECG measurement gaps and risk of missing remarkable heart events, restricting the daily activities of the user and a poor user acceptance.

Document U52020144777 discloses a textile interconnection system for a textile substrate containing conductive fibers configured to transmit at least one of a power or data signal. The textile interconnection system includes a textile receptacle projecting from the textile substrate to define a cavity for receiving a controller device. A textile docking device is received within the textile receptacle and coupled to the at least one conductive fiber of the textile substrate to electrically interconnect the received controller device and the textile substrate. A housing is coupled to the textile docking device and received within the textile receptacle to mechanically interconnect the received controller device and the textile substrate. The textile interconnection system is complex, notably due to the need of the textile docking device present on the textile.

Document US2014187899 discloses an electrode assembly including a thin, low thickness snap for heart rate monitoring. The electrode assembly includes two electrodes with respective snaps connected by a non-stretchable material portion. The electrode assembly can be in the form of a kit with multiple pieces or in an assembled form. The arrangement with integrated electrodes is not adapted for other electronic equipment to connect to the garment, and cannot provide non-defective electric connection with the conductive part of the garment.

Document US 2015119677 relates to an article of clothing and ECG electrodes, including a front piece and a back piece. The front piece includes an inner lining and an outer lining. Ten front-end modules of active electrodes, ten lead wires, and an electrical plug are disposed on the outer surface of the inner lining for the purpose of collecting electrical signals. The ten front-end modules are connected to the electrical plug via the ten lead wires, respectively. The front-end modules are riveted on the inner lining by bolts. The outer lining includes a pocket including a through hole. The electrical plug passes through the through hole and is inserted into a Holter system placed in the pocket. A plurality of female buckles for buckling ECG electrodes are disposed on an inner surface of the inner lining. The electrode is integrated to the connecting system and non-detachable to be changed or to mount another electronic equipment to the conductive part of the article of clothing.

Document U.S. Pat. No. 9,326,695 concerns an electrode harness comprising a non-releasable connector with a female orifice and a bottom member having a dry electrode and a male locking stud with a sharpened penetrator for piercing the electrode harness. The top and bottom members are attached by a connecting bridge. The electrode harness is not allows for other electronic equipment to connect to the harness, and does not provide non-defective electric connection with the conductive part of the harness.

### SUMMARY

An aim of the present invention is the provision of a connector assembly that overcomes the shortcomings and limitations of the state of the art.

The present disclosure concerns a sensing connector assembly for providing an electrical connection between an electrical contact of a sensor device and an electrically conductive path on a first side of a wearable fabric, the sensing connector assembly comprising: a first connecting part destined to contact the first side and comprising an opening configured to receive the sensor device; a second connecting part destined to contact a second side of the wearable fabric, the second connecting part being configured to mechanically cooperate with the first connecting part such that the first and second connecting parts can be fastened on each side of the wearable fabric and extend in a connection plane. The first connecting part comprises a first connecting rim at the periphery of the opening, the connecting rim being configured to cooperate with sensor device such as to attach the sensor device to the first connecting part and such that the electrical contact exerts a predetermined force in a direction substantially orthogonal to the connection plane and against the first side of the wearable fabric, when the second connecting part is fastened to the first connecting part.

The present disclosure further concerns a system comprising a wearable fabric including at least an electrically conductive path and at least one sensing connector assembly.

The sensing connector assembly allows to connect in a reliable fashion a sensor device on a piece of garment comprising a conductive path. Other advantages of the sensing connector assembly and system are mentioned in the description.

### SHORT DESCRIPTION OF THE DRAWINGS

Exemplar embodiments of the invention are disclosed in the description and illustrated by the drawings in which:

FIG. 1 shows a cross section view of a sensor connector assembly comprising a first connecting part and a second connecting part, according to an embodiment;

FIG. 2 shows an exploded view of the sensor connector assembly, according to an embodiment;

FIG. 3 shows a cross section view of the first connecting part fastened to the second connecting part, according to an embodiment;

FIG. 4 shows a cross section view of the sensor device, according to an embodiment;

FIG. 5 shows a view of a sensor device on the side of electrical contact, according to an embodiment;

FIG. 6 represents a conductive path on a wearable fabric, according to an embodiment;

FIG. 7 shows a system comprising a plurality of the sensor connector assembly for monitor physical parameters of a user, according to an embodiment;

FIG. 8 represents a cross section view showing a portion of the system, according to an embodiment;

FIG. 9 shows details of conductive path on a wearable fabric; and

FIG. 10 shows an alternative configuration of the sensing connector assembly.

### EXAMPLES OF EMBODIMENTS

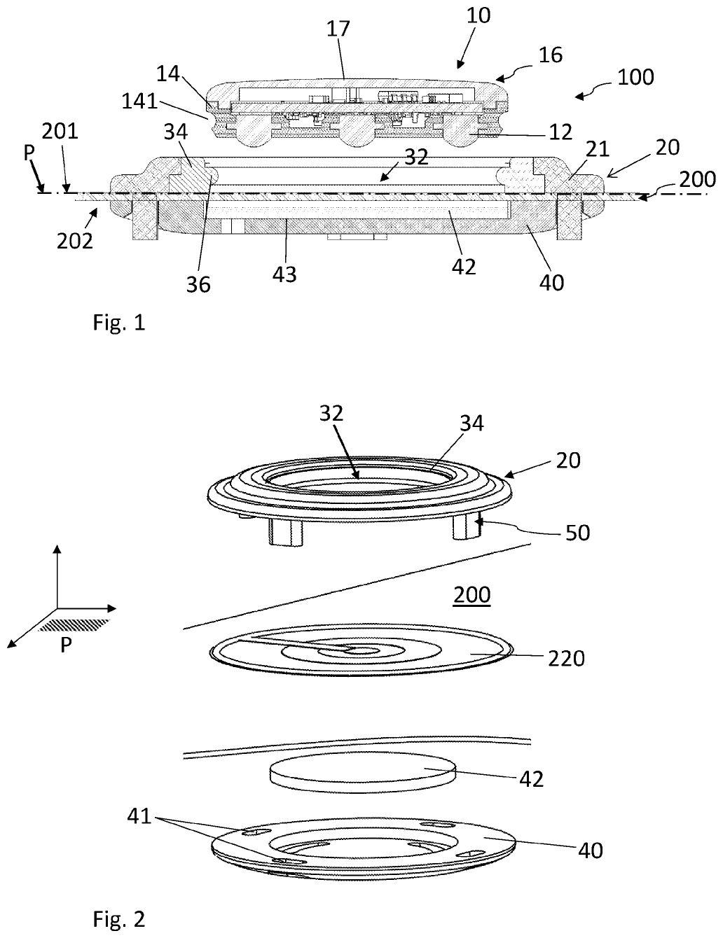

FIG. 1 shows a cross section view of a sensor connector assembly 100 according to an embodiment. The sensor connector assembly 100 is configured for providing an electrical connection between an electrical contact 12 of a sensor device 10 and an electrically conductive path 220 on a first side 201 of a wearable fabric 200. The sensor connector assembly 100 comprises a first connecting part 20 destined to contact the first side 201. The first connecting part 20 comprises an opening 32 configured to receive the sensor device 10. The sensor connector assembly 100 further comprises a second connecting part 40 destined to contact a second side 202 of the wearable fabric 200. The second connecting part 40 is configured to mechanically cooperate with the first connecting part 20 such that the first and second connecting parts 20, 40 can be fastened on each side 201, 202 of the wearable fabric 200 and extend in a connection plane P.

The first connecting part 20 comprises a first connecting rim 34 at the periphery of the opening 32. The connecting rim 34 is configured to cooperate with sensor device 10 such as to attach the sensor device 10 to the first connecting part 20 and such that the electrical contact 12 exerts a predetermined force in a direction substantially orthogonal to the connection plane P and against the first side 201 of the wearable fabric 200, when the second connecting part 40 is fastened to the first connecting part 20. In one aspect, the predetermined force can be between 10 gf and 300 gf (0.098 N and 2.94 N) per electrical contact 12.

In the configuration shown in FIG. 1, the first connecting part 20 is ring shaped and the opening 32 is substantially circular. The sensor device 10 is disc shaped having a diameter such as to fit in the circular opening 32.

In one aspect, the electrical contact comprises at least one connection pin 12 protruding from the sensor device 10 in a direction substantially orthogonal to the connection plane P.

In one aspect, the sensor device 10 comprises a second connecting rim 14 configured to cooperate with the first connecting rim 34 such as to provide a water-tight fitting connection between the sensor device 10 and the first connecting part 20. As illustrated in FIG. 1, the first connecting part 20 is form from a frame portion 21 and the first connecting rim 34. The frame portion 21 and the first connecting rim 34 can be two separate parts or made integral (as a single part).

The first connecting rim 34 can comprise a projecting edge 36 and the second connecting rim 14 can comprise a grooved edge substantially conformal with the projecting edge 36, such as to form a water-tight form-fitting connection between the sensor device 10 and the first connecting part 20. The projecting edge 36 and the first connecting rim 34 can be two separate parts or made integral (as a single part).

In the variant illustrated in FIG. 1, the first connecting rim 34 comprises a rib and the second connecting rim 14 comprises a groove 140. However, other configurations of the first and second connecting rims 34, 14 can be contemplated. For example, in a variant not represented, the first connecting rim 34 can comprise a groove and the second connecting rim 14 can comprise a rim.

The projecting edge 36 can be made of a flexible material, such as a rubber like material. The projecting edge 36 can be more flexible than the first connecting rim 34 and the frame portion 21. Alternatively, the first connecting rim 34 and the projecting edge 36 can be more flexible than the frame portion 21. In a possible configuration, the first connecting rim 34 is over-molded on the frame portion 21.

In an embodiment, the second connecting part 40 comprises an elastic element 42 arranged to contact the second side 202 when the second connecting part 40 is fastened to the first connecting part 20.

In one aspect, the second connecting part 40 can comprise a recess 43 configured to accommodate the elastic portion 42. The recess 43 is provided on the side of the second connecting part 40 facing the first connecting part 20 when the second connecting part 40 is fastened to the first connecting part 20. In the specific example of FIG. 1, the recess 43 and the elastic portion 42 are disc shaped. Other configurations of the recess 43 and the elastic portion 42 can be contemplated, for example the depth of the recess 43 and the thickness of the elastic portion 42, as long as the elastic portion 42 can contact the wearable fabric 200 when the second connecting part 40 is fastened to the first connecting part 20.

The elastic portion 42 can be made of a single elastic element (for example the disc shown in FIG. 1) or can comprise a plurality of elements. The elastic portion 42 can be made of a foam such as a polyester foam, natural rubber (NR), styrene-butadiene rubber (SBR) or polyurethane foam. Preferably, the elastic portion 42 can have a hardness of about 28 and 42, for example 35, measured with a Shore durometer by using the A scale.

FIG. 2 shows an exploded view of the sensor connector assembly 100 according to an embodiment. The first connecting part 20 comprises a coupling element 50 configured to pass through the wearable fabric 200 and cooperate with a receiving element 41 of the second connecting part In the example illustrated in FIG. 2, the coupling element comprises a plurality of legs 50 extending substantially perpendicular to the connection plane P. The receiving element comprises a plurality of slots 41 provided in the second connecting part 40, each slot 41 cooperating with a leg 50 for fastening the first connecting part 20 to the second connecting part 40. Here, the slots 41 are through holes and the legs can be glued, soldered or over-molded, in order to be fix the second connecting part 40. The legs 50 pass through the wearable fabric 200 by piercing the latter or through holes (not shown) provided in the wearable fabric 200.

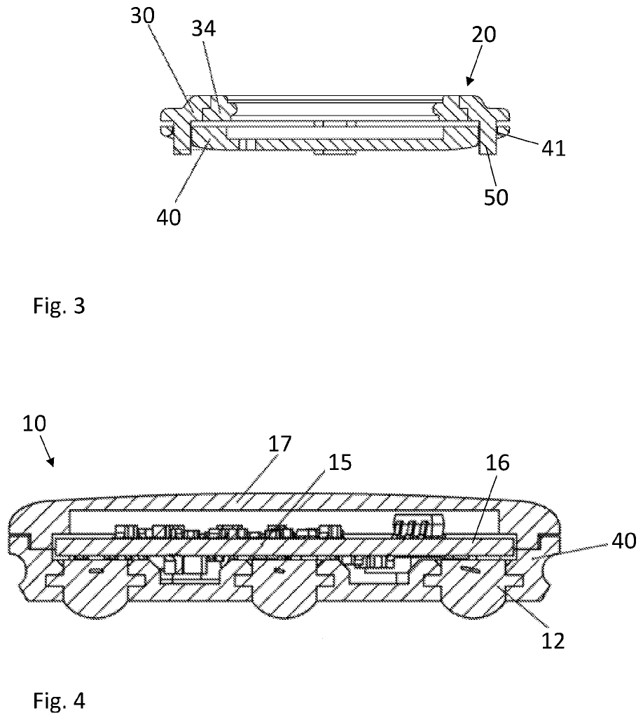

FIG. 3 shows a cross section view of the first connecting part 20 fastened to the second connecting part 40, where the legs 50 extending through the slots 41. Other configurations of the coupling element 50 and the receiving element 41 are possible. In an non illustrated example, the legs 50 can include a step portion at its distal end such as to form an attachment clip. The leg 50 can be pushed into the through-hole slot 41 and the step portion retains the leg 50 within the slot 41. In other possible configurations, the coupling element 50 can comprise a rivet.

Once the first connecting part 20 is fastened to the second connecting part 40 across the wearable fabric 200, the first connecting part should be located where an electrically conductive path 220 is present. The conductive path 220 is then accessible within the opening 32 of the first connecting part 20 to the sensor device 10.

FIG. 4 shows a cross section view of the sensor device 10, according to an embodiment. Here, the sensor device 10 comprises an electrode sensor comprising an electrode 17 on the side of the electrode sensor 10 opposed to the one comprising the electrical contact 12. The electrode 17 is configured to contact the skin of the user when the first connecting part 20 is fastened to the second connecting part 40 across the wearable fabric 200 and the wearable fabric 200 is worn by the user. In the example of FIG. 4, the electrode 17 is disk shaped and slightly cambered such as to maximize contact with skin toward the center of the electrode 17. The electrode 17 can be electrically connected to a PCB 16 comprising an electronic circuit 15 connected to the electrical contact 12 destined to contact the conductive path 220.

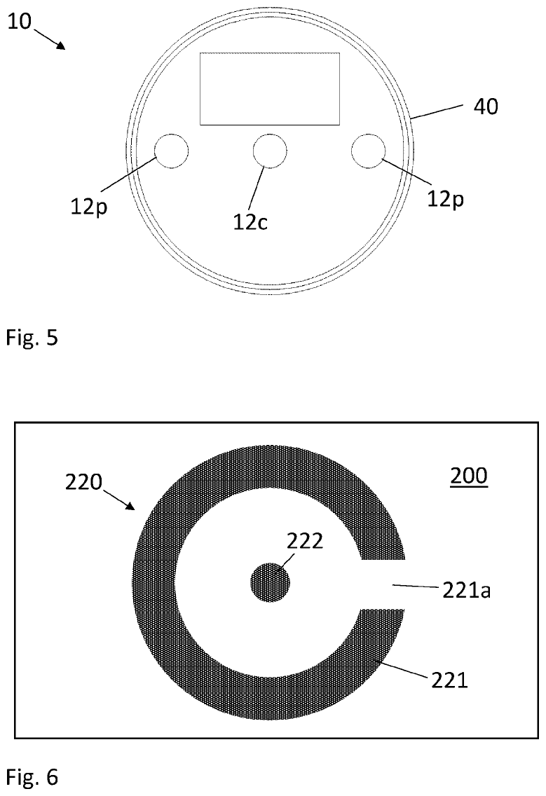

In one aspect, the electrical contact comprises three pins 12. The three pins 12 can be aligned as illustrated in FIG. 5 showing a view of the sensor device 10 on the side of the electrical contact 12. Such arrangement of the pins 12 can be advantageous for contacting a conductive path 220 such as shown in FIG. 2. More particularly, the conductive path 220 comprises a ring-shaped outer contact 221 (see FIG. 6) and a disc-shaped inner contact 222. When the first connecting part 20 is fastened to the second connecting part 40 across the wearable fabric 200, the central pin 12c contacts the inner contact 222 and the peripheral pins 12p contact the outer contact 221. The outer contact 221 may comprise a non-electrically conducting portion 221a due to fabrication constrains. The two peripheral pins 12p allows to insure an electrical contact between the sensor device 10 and the outer contact 221 independently of the angular orientation of the sensor device 10 when the latter is attached to the first connecting part 20. Such configuration of the electrical contact 12 allows to freely rotate the sensor device 10 relative to the first connecting part 20 without losing the electrical contact with the conductive path 220. Other configuration of the electrical contact 12 can be considered, such as a different number of pins, a different arrangement of the pins.

As mentioned above, the connecting rim 34 is configured such that the electrical contact 12 exerts a predetermined force, such as between 10 gf and 300 gf (0.098 N and 2.94 N) per electrical contact 12, in a direction substantially orthogonal to the connection plane P and against the first side 201 of the wearable fabric 200, when the second connecting part 40 is fastened to the first connecting part 20. Thus, when the sensor device 10 is attached to the first connecting part 20, the pins 12 (12c, 12p) exerts the predetermined force on the conductive path 220. The predetermined force further depends on the elastic portion 42 on the opposite side of the wearable fabric 200. The elastic portion 42 provides a spring effect that can permit to retain the conductive path 220 in position and in physical contact relative to the electrical contact 12, ensuring the electric contact between the sensor device 10 and the conductive path 220. The elastic portion 42 can further uniformize the force along the conductive path 220 and the electrical contact 12.

Other configurations of the sensor device 10 are contemplated. In one aspect not illustrated, the sensor device 10 comprises an electrode sensor including no electronic circuit. The electrode sensor 10 can include several electrodes on the side of the electrode sensor 10 opposed to the electrical contact 12. In another aspect, the sensor device 10 can comprise an electronic circuit to control an electrode sensor and a powering means to power the sensor device.

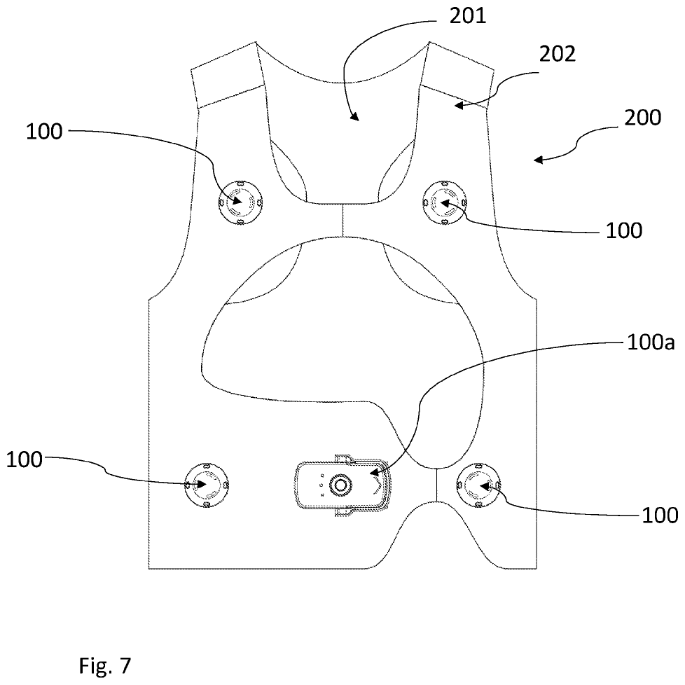

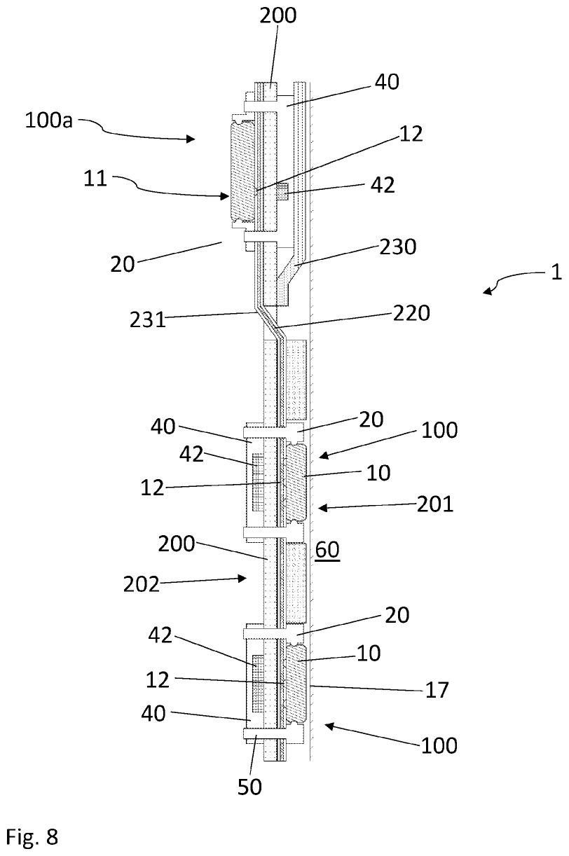

A system 1 for monitor physical parameters of a user is shown in FIG. 7, according to an embodiment. The system 1 comprises the wearable fabric 200 adapted to be worn by a user and having at least an electrically conductive path (not shown in FIG. 7) on a first side 201. The system 1 further comprises at least one sensing connector assembly 100, wherein the second connecting part 40 is fastened to the first connecting part 20. In the configuration shown in FIG. 7, the system 1 comprises five sensing connector assemblies 100. Four sensing connector assemblies 100 are configured to attach an electrode sensor 10 and one 100a of the sensing connector assemblies 100 is configured to attach a central electronic unit 11 (see FIG. 8). The central electronic unit 11 can be configured to power and control the electrode sensor 10 of the sensing connector assembly 100.

FIG. 8 represents a cross section view showing a portion of the system 1, according to an embodiment. In FIG. 8, two sensing connector assemblies 100 comprising an electrode sensor 10 and one sensing connector assembly 100a comprising central electronic unit 11 is represented. FIG. 8 also shows the conductive path 220 on the first side 201 of the wearable fabric 200. The sensing connector assemblies 100, 100a are shown when the second connecting part 40 is fastened to the first connecting part 20 across the wearable fabric 200 and the electrical contact 12 contacting the conductive path 220. When the wearable fabric 200 is worn by the user, the electrode 17 of the electrode sensor 10 is in contact with the user's body 60, typically the electrode 17 is in contact with the user's skin.

The sensing connector assembly 100a comprising the central electronic unit 11 can be fastened on the wearable fabric 200 in an opposed fashion to the connector assembly 100 comprising the electrode sensor 10. In other words, the first connecting part 20 is mounted on the first side 201 and the second connecting part 40 is mounted on the second side 202 of the wearable fabric 200. In such configuration, the central electronic unit 11 is on the first side 201 of the wearable fabric 200 and cannot come in contact with the user's body 60 when the wearable fabric 200 is worn. A protective fabric layer 230 can be inserted on the external surface of the second connecting part 40 of the sensing connector assembly 100a comprising the central electronic unit 11 such that the protective fabric layer 230 is between the second connecting part 40 and the user's body 60 when the wearable fabric 200 is worn. An insulating layer 231 can further be inserted on top of the conductive path 220 such as to electrically insulated the user's body 60 from the conductive path 220 when the wearable fabric 200 is worn.

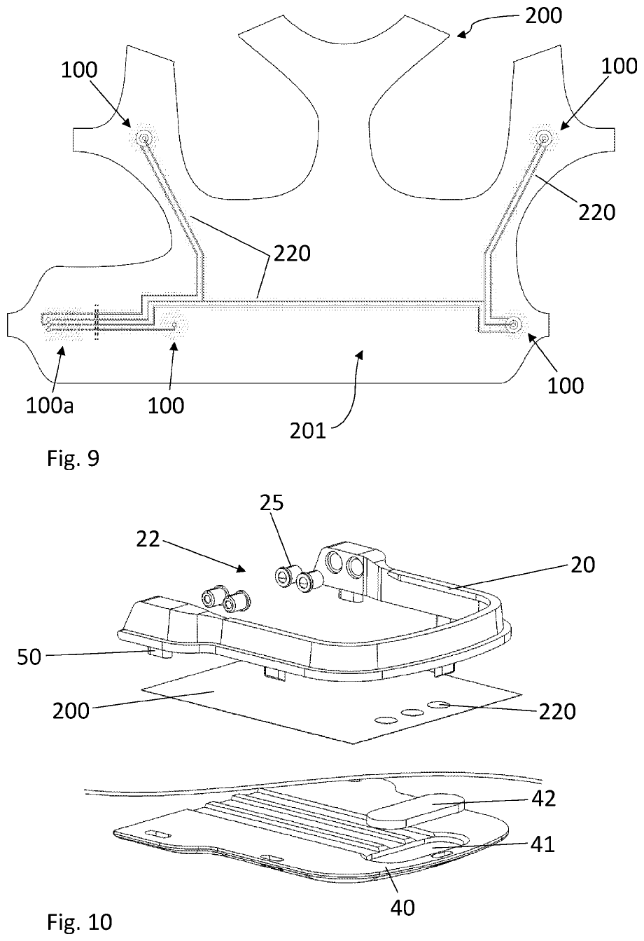

FIG. 9 shows details of the conductive path 220 on the wearable fabric 200. The conductive path 220 can comprise one or a plurality of electrically conductive wires, or tracks, connecting the different electrode sensors 10 to the central electronic unit 11.

The conductive path 220 can be printed, deposited, or can comprise electrically conductive yarns sewn or woven onto the fabric substrate. The conductive path 220 can be used for transmitting electrical signals, information signals and/or power signals. Transmission of information signals can be achieved by using multi-communication stream using different frequencies. For example, a single wire can be used to transmit simultaneously multiple signals, each signal having a different frequency band, for instance between 1 k and 1 MHz.

The wearable fabric 200 can comprise a piece of garment such as a vest, a bra or a shirt. The wearable fabric 200 can further comprise a harness, a chest band or an armband.

The wearable fabric 200 can be provided with the conductive path 220 and the first connecting part 20 as a reusable or disposable system.

FIG. 10 shows an alternative configuration of the sensing connector assembly 100 wherein the first connecting part 20 has an open geometry. The open geometry of the first connecting part 20 allows to insert the sensor device 10 to be attached to the first connecting part 20 from the open side 22 of the first connecting part 20. The first connecting part 20 can comprise rollers 25 for guiding the sensor device 10 when inserted into the first connecting part 20. The configuration of the sensing connector assembly 100 shown in FIG. 10 can be used advantageously for attaching the central electronic unit 11. Note that other configurations of the sensing connector assembly 100 are also possible.

The first connecting part 20 and/or the second connecting part 40 can be over-molded on the wearable fabric 200.

The system 1 can form a three-lead ECG measuring system, or standard three-lead configuration.

Advantages of the system 1 disclosed herein include the use of dry electrodes, allowing the monitoring for a long period of time (minimum 2 weeks or months). The system 1 does not cause hindrance to user movements, can be easily worn and can be adapted to any fashion. It is adapted for all age groups. The system 1 does not require the use of cables.

The system 1 can be further used for other applications than ECG measuring or monitoring, such as measuring/monitoring SpO₂, blood pressure, body temperature or any other vital sign parameters or signals to be detected outside a user's body as e.g. room temperature, gas concentration (CO₂, CO, O₂), proximity, etc.

The system 1 can be worn by a user (person, namely a human being or an animal) to monitor or measure the above physical parameters in relation with the user's body or in relation with the environment of the user.

### REFERENCE NUMERAL USED IN THE FIGURES

1 system

10 sensor device, electrode sensor

11 central electronic unit

12 electrical contact

14 second connecting rim

141 groove

15 electronic circuit

16 PCB

17 electrode

20 first connecting part

21 frame portion

22 open side

25 roller

32 opening

34 first connecting rim

36 projecting edge

40 second connecting part

41 receiving element, slot

42 elastic portion (foam pad)

43 recess

50 joining element

60 body

100, 100a sensing connector assembly

200 wearable fabric

201 first side

202 second side

220 conductive path

221 outer contact

221a non-electrically conducting portion

222 inner contact

230 protective fabric layer

231 insulating layer

P connection plane

1. Sensing connector assembly for providing an electrical connection between an electrical contact of a sensor device and an electrically conductive path on a first side of a wearable fabric, the sensing connector assembly comprising:
a first connecting part destined to contact the first side of the wearable fabric and comprising an opening configured to receive the sensor device;
a second connecting part destined to contact a second side of the wearable fabric, the second connecting part being configured to mechanically cooperate with the first connecting part such that the first and second connecting parts can be fastened on each side of the wearable fabric and extend in a connection plane;
wherein the first connecting part comprises a first connecting rim at the periphery of the opening, the connecting rim being configured to cooperate with sensor device, wherein the sensor device comprises a second connecting rim configured to cooperate with the first connecting rim and wherein the first connecting rim comprises a projecting edge and the second connecting rim comprises a grooved edge substantially conformal with the projecting edge, such as to attach the sensor device to the first connecting part such as to form a water-tight form-fitting connection, and such that the electrical contact exerts a predetermined force in a direction substantially orthogonal to the connection plane and against the first side of the wearable fabric, when the second connecting part is fastened to the first connecting part.

2. Sensing connector assembly according to claim 1, wherein the second connecting part comprises an elastic element arranged to contact the second side when the second connecting part is fastened to the first connecting part.

3. Sensing connector assembly according to claim 1, wherein the electrical contact comprises at least one connection pin protruding from the sensor device in a direction substantially orthogonal to the connection plane.

4. (canceled)

5. (canceled)

6. Sensing connector assembly according to claim 5, wherein the first connecting rim comprises a rib and the second connecting rim comprises a groove.

7. Sensing connector assembly according to claim 1, wherein the first connecting part comprises a coupling element configured to pass through the wearable fabric and cooperate with a receiving element of the second connecting part.

8. Sensing connector assembly according to claim 1,
wherein the sensor device comprises an electrode sensor comprising an
electrode on the side of the electrode sensor opposed to the one comprising the electrical contact.

9. Sensing connector assembly according to claim 1,
wherein the sensor device comprises a central electronic unit configured to power and control electrode sensors.

10. System comprising a wearable fabric adapted to be worn by a user and having at least an electrically conductive path on a first side (204), the system further comprising at least one sensing connector
assembly, the sensing connector assembly comprising a first connecting part destined to contact the first side of the wearable fabric and comprising an opening configured to receive the sensor device;
a second connecting part destined to contact a second side of the wearable fabric, the second connecting part being configured to mechanically cooperate with the first connecting part such that the first and second connecting parts can be fastened on each side of the wearable fabric and extend in a connection plane;
wherein the first connecting part comprises a first connecting rim at the periphery of the opening, the connecting rim being configured to cooperate with sensor device,
wherein the sensor device comprises a second connecting rim configured to cooperate with the first connecting rim, and
wherein the first connecting rim comprises a projecting edge and the second connecting rim comprises a grooved edge substantially conformal with the projecting edge, such as to attach the sensor device to the first connecting part such as to form a water-tight form-fitting connection, and such that the electrical contact exerts a predetermined force in a direction substantially orthogonal to the connection plane and against the first side of the wearable fabric, when the second connecting part is fastened to the first connecting part,
wherein the second connecting part is fastened to the first connecting part.

11. System according to claim 8,
comprising at least two sensing connector assemblies,
wherein the sensor device of at least one sensing connector assembly comprises a central electronic unit and the sensor device of at least one sensing connector assembly comprises an electrode sensor comprising an electrode on the side of the electrode sensor opposed to the one comprising the electrical contact, the central electronic unit being configured to power and control the electrode sensor.

12. System according to claim 9,
wherein the electrode is configured to contact the skin of the user when the wearable fabric is worn by the user.

13. System according to claim 8, wherein the wearable fabric comprises a vest, bra, a shirt, a chest band or armband.

14. System according to claim 8, forming a three-lead ECG measuring system.

15. System according to claim 8, wherein said predetermined force is between 0.098 N and 2.94 N per electrical contact.
